(19) United States
(12) Patent Application Publication
(10) Pub. No.: US 2020/0306171 A1
(43) Pub. Date: Oct. 1, 2020

(54) PERSONAL CARE AEROSOL COMPOSITIONS, METHODS AND DEVICES

(71) Applicant: HONEYWELL INTERNATIONAL INC., MORRIS PLAINS, NJ (US)

(72) Inventors: Barry Setiawan, Morris Plains, NJ (US); Yiu Keung Ling, Morris Plains, NJ (US); Anthony Anzalone, Morris Plains, NJ (US); Prema Khanka-Bohra, Morris Plains, NJ (US)

(21) Appl. No.: 16/825,301

(22) Filed: Mar. 20, 2020

Related U.S. Application Data
(60) Provisional application No. 62/827,697, filed on Apr. 1, 2019.

(51) Int. Cl. A61K 8/92; A61K 8/04; A61K 8/96; A61K 8/31; A61Q 15/00

(52) U.S. Cl. CPC A61K 8/922; A61K 8/046; A61Q 15/00; A61K 8/315; A61K 8/965

(57) ABSTRACT

Disclosed are personal care formulations for delivery to the skin of a person in the form of an aerosol comprising: (1) non-propellant components comprising at least one active ingredient in the form of finely divided particles, at least one hydrophobized clay, a hydrophobic carrier comprising trans-1-chloro-3,3,3-trifluoropropene (trans-1233zd) and at least one co-carrier wherein: (i) the weight ratio of said trans-1233zd:co-carrier is from about 0.25:1 to about 4:1; (ii) said trans-1233zd and said co-carrier together comprise at least about 40% by weight of said hydrophobic carrier.

This application is related to and claims the priority benefit of U.S. Provisional Application 62/827,697, filed on Apr. 1, 2019, which is incorporated herein by reference.

### FIELD OF THE INVENTION

### Field of the Invention

This invention relates generally to aerosols and to methods and devices for delivering aerosols to the skin and/or hair of a person, including to antiperspirant compositions and other personal care compositions for delivery via aerosols of the powder suspension type suitable for spraying from a pressurized container.

### Background

Antiperspirant compositions normally contain an antiperspirant active compound which chemically suppresses the production of perspiration by sweat glands. One example of such a compound is aluminum chlorohydrate, which acts as an astringent to inhibit perspiration of the user. Such antiperspirant formulations can be applied to the skin using several different modes of application, including roll-on and stick forms. However, those skilled in the art will appreciate that applying an antiperspirant formulation in the form of an aerosol spray can have several advantages when applied on the skin.

For aerosol antiperspirant sprays, the antiperspirant active compound is usually suspended as an insoluble powder in a liquid carrier together with a non-aqueous liquefied volatile propellant in a pressurized aerosol container. The aerosol spray is produced by rapid transition of the propellant from the liquid to the vapor phase as it dispenses from the atomizing valve of the aerosol container. As a result, application of antiperspirant using an aerosol spray has gained wide acceptance for a number of reasons. Aerosol application of an antiperspirant active compound in the form of a dry powder suspended in the propellant is cosmetically desirable because the antiperspirant can be smoothly and effectively applied to the skin in a manner that feels dry and comfortable. Furthermore, because the antiperspirant powder does not dissolve in the liquefied propellant medium, the antiperspirant salt does not corrode ordinary metal aerosol cans.

One difficulty associated with antiperspirant aerosol sprays is that the delivery of small particles of antiperspirant active compound can pose some technical difficulties. For example, a finely divided spray can create difficulties in ensuring that a large proportion of the active components travel to, remain on and are absorbed by the skin of the user. This problem arises because the dry powder spray in many cases tends to form a wide dispersion or cloud, resulting in many of the active particles not hitting the target area. Also, even when the particles hit the target area, the relatively high energy imparted to the particles by the propellant can cause many particles to bounce off the skin of the user. In order to address these problems, it has been common practice to utilize an anhydrous liquid carrier component to disperse and/or suspend the active powder, and then fine droplets of the anhydrous carrier are able to travel to the target area in a more well-formed spray pattern, and the problem of dry particles bouncing off the user is greatly reduced. Certain silicone liquids have been heretofor developed for use as carrier fluids for the active ingredients in several personal care compositions, including in antiperspirant aerosol sprays. However, a need has developed to reduce or eliminate the use of the frequently used volatile silicone fluids “D-4,” which is octamethylcyclo-tetrasiloxane [(CH3)2SiO]4 and “D-5,” which is decamethylcyclopentasiloxane [(CH3)2SiO]5, in products which result in those liquids escaping into the biosphere. For example, see the publication by the Danish Ministry of the Environment has published a paper entitled “Siloxanes (D3, D4, D5, D6, HMDS) Evaluation of health hazards and proposal of a health-based quality criterion for ambient air Environmental Project No. 1531, 2014), which is incorporated herein by reference. Developing a substitute carrier liquid for the silicone liquids used for the active ingredients in personal care products, including antiperspirant aerosol spray products, represents significant technical challenges. For example, the substitute fluid must be able to effectively disperse and/or suspend the active compounds without deteriorating the effectiveness of the particles for the intended purpose, such as to prevent perspiration. In addition, the carrier fluid must satisfy a difficult-to-achieve combination of features, including the ability to remain as a fine, well-targeted spray as it travels to the skin of the user and at the same time contact the skin without imparting either an overly wet/oily feel or an overly dry feel. It is a difficult technical challenge to develop a new carrier fluid that is able to at once achieve this combination of features at the same level as has been achieved by the previously used silicone liquids.

### SUMMARY

The present invention thus provides a personal care formulation for delivery to the skin of a person in the form of an aerosol comprising:

(1) non-propellant components comprising:

(a) from about 0.5% to about 80% by weight of at least one active ingredient in the form of finely divided particles;

(b) from about 0.5% to about 15% by weight of at least one hydrophobized clay;

(c) from about 10% to about 85% by weight of a hydrophobic carrier comprising trans-1-chloro-3,3,3-trifluoropropene (trans-1233zd) and at least one co-carrier wherein: (i) the weight ratio of said trans-1233zd:co-carrier is from about 0.25:1 to about 4:1; (ii) said trans-1233zd and said co-carrier together comprise at least about 40% by weight of said hydrophobic carrier; and (iii) said co-carrier is of a type and present in an amount to provide said formulation with a dry silky feel when sprayed in the form of an aerosol onto the skin of the person; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d).

For the purposes of convenience, personal care formulations according to this paragraph are referred to herein as Formulation 1.

As used herein, “active ingredient” means a compound or compounds that have or are intended to have an impact on the personal care of the user, including but not limited to the feel, moisture content, texture, shape, color or health of the skin or hair.

As used herein, “dry silky feel” means a feeling on the skin or scalp that is not overly wet or overly dry as those terms are understood in the industry.

As used herein, the term “interfaced” with means that the propellant is at least in contact with the non-propellant components and is able to directly exert a propelling force on the non-propellant components.

As used herein, “separated from” means that a wall, and preferably a flexible wall, separates the non-propellant components from at least a portion of the propellant, as would be the case for example of a formulation contained in a bag-on-valve arrangement.

As used herein, the term “carrier” refers to components of the formulation which make up the continuous liquid phase of the formulation (exclusive of any propellant that may be present in the liquid phase) plus any components that are present in the continuous liquid phase for the purpose of aiding in the suspension and/or stabilizing the dispersion of the active ingredient.

The present invention also provides a personal care formulation for delivery in the form of an aerosol to the skin of a person comprising:

(1) non-propellant components comprising:

(a) from about 0.5% to about 80% by weight of at least one active ingredient in the form of finely divided particles;

(b) from about 0.5% to about 15% by weight of at least one hydrophobized clay;

(c) from about 10% to about 85% by weight of a hydrophobic carrier comprising trans-1-chloro-3,3,3-trifluoropropene (trans-1233zd) and at least one co-carrier comprising one or more of: (i) triglycerides of linear or branched, saturated or unsaturated, C8-30 fatty acids; (ii) esters of at least one C2-C9 monocarboxylic, dicarboxylic or tricarboxylic acid and at least one linear or branched, saturated or unsaturated alcohol having 1 to 30 carbon atoms; (iii) C10-C20 iso paraffin; and (iv) volatile silicone fluids other than D-4 and D-5, wherein the weight ratio of said trans-1233zd:co-carrier is from about 0.25:1 to about 4:1 and said trans-1233zd and said co-carrier together comprise at least about 40% by weight of said hydrophobic carrier; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d).

For the purposes of convenience, personal care formulations according to this paragraph are referred to herein as Formulation 2.

The present invention also provides a personal care formulation for delivery in the form of an aerosol comprising:

(1) non-propellant components comprising:

(a) from about 1% to about 60% by weight of at least one at least one active ingredient;

(b) from about 0.5% to about 15% by weight of at least one hydrophobized clay;

(c) from about 10% to about 85% by weight, based on the total weight of the components in the formulation, of a hydrophobic carrier comprising trans-1-chloro-3,3,3-trifluoropropene (trans-1233zd) and at least one co-carrier comprising one or more of: (i) triglycerides of linear or branched, saturated or unsaturated, C8-30 fatty acids; and (ii) esters of at least one C2-C9 monocarboxylic, dicarboxylic or tricarboxylic acid and at least one linear or branched, saturated or unsaturated alcohol having 1 to 30 carbon atoms, wherein the weight ratio of said trans-1233zd:co-carrier is from about 0.25:1 to about 4:1 and wherein said trans-1233zd and said co-carrier together comprise at least about 40% by weight of said hydrophobic carrier; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d).

For the purposes of convenience, personal care formulations according to this paragraph are referred to herein as Formulation 3.

The present invention also provides personal care formulations for delivery in the form of an aerosol comprising:

(1) non-propellant components comprising:

(a) from about 1% to about 60% by weight of at least one active ingredient;

(b) from about 0.5% to about 15% by weight of at least one hydrophobized clay;

(c) from about 10% to about 85% by weight of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of one or more triglycerides of linear or branched, saturated or unsaturated, C8-30 fatty acids, wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with or separated from said components (a)-(d).

For the purposes of convenience, personal care formulations according to this paragraph are referred to herein as Formulation 4.

The present invention also provides personal care formulations for delivery in the form of an aerosol comprising:

(1) non-propellant components comprising:

(a) from about 1% to about 60% by weight of at least one perspiration-inhibiting antiperspirant active ingredient;

(b) from about 0.5% to about 15% by weight of at least one hydrophobized clay;

(c) from about 10% to about 85% by weight of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of one or more triglycerides that are natural oils, wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with or separated from said components (a)-(d).

For the purposes of convenience, personal care formulations according to this paragraph are referred to herein as Formulation 5.

As used herein, “natural oil” refers to oils that are extracted from naturally occurring plant material.

The present invention also provides personal care formulations for delivery in the form of an aerosol comprising:

(1) non-propellant components comprising:

(a) from about 1% to about 60% by weight of at least one active ingredient;

(b) from about 0.5% to about 15% by weight of at least one hydrophobized clay;

(c) from about 10% to about 85% by weight of a hydrophobic carrier comprising at least about 20% by weight of all the carrier components of trans-1-chloro-3,3,3-trifluoropropene (trans-1233zd) and at least about 20% by weight of all the carrier components a first co-carrier selected from the group consisting of consisting of soy bean oil, cottonseed oil, sunflower oil, palm oil, palm kernel oil, linseed oil, almond oil, castor oil, corn oil, olive oil, rapeseed oil, sesame oil, safflower oil, wheat germ oil, peach kernel oil, the liquid components of coconut oil and the combination of two or more of these, wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with or separated from said components (a)-(d).

For the purposes of convenience, personal care formulations according to this paragraph are referred to herein as Formulation 6.

The present invention also provides personal care formulations for delivery in the form of an aerosol comprising:

(1) non-propellant components comprising:

(a) from about 1% to about 60% by weight of at least one active ingredient;

(b) from about 0.5% to about 15% by weight of at least one hydrophobized clay;

(c) from about 10% to about 85% by weight of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of safflower oil, wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with or separated from said components (a)-(d).

For the purposes of convenience, personal care formulations according to this paragraph are referred to herein as Formulation 7.

The present invention also provides personal care formulations for delivery in the form of an aerosol comprising:

(1) non-propellant components comprising:

(a) from about 1% to about 60% by weight of at least one active ingredient;

(b) from about 0.5% to about 15% of at least one hydrophobized clay;

(c) from about 10% to about 85% by weight of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of esters of at least one C2-C7 monocarboxylic, dicarboxylic or tricarboxylic acid and at least one linear or branched, saturated or unsaturated alcohol having 1 to 30 carbon atoms, wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d). For the purposes of convenience, personal care formulations according to this paragraph are referred to herein as Formulation 8.

The present invention also provides personal care formulations for delivery in the form of an aerosol comprising:

(1) non-propellant components comprising:

(a) from about 1% to about 60% by weight of at least one active ingredient;

(b) from about 0.5% to about 15% of at least one hydrophobized clay;

(c) from about 10% to about 85% by weight of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of diesters of at least one C2-C7 dicarboxylic or tricarboxylic acid and at least one linear or branched, saturated or unsaturated alcohol having 4 to 10 carbon atoms wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d). For the purposes of convenience, personal care formulations according to this paragraph are referred to herein as Formulation 9.

The present invention also provides personal care formulations for delivery in the form of an aerosol comprising:

(1) non-propellant components comprising:

(a) from about 1% to about 60% by weight of at least one active ingredient;

(b) from about 0.5% to about 15% of at least one hydrophobized clay;

(c) from about 10% to about 85% by weight of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of diesters of at least one C3-C5 dicarboxylic or tricarboxylic acid and at least one linear or branched, saturated or unsaturated alcohol having 5 to 9 carbon atoms wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d). For the purposes of convenience, personal care formulations according to this paragraph are referred to herein as Formulation 10.

The present invention thus provides an antiperspirant formulation for delivery to the skin of a person in the form of an aerosol comprising:

(1) non-propellant components comprising:

(a) from about 0.5% to about 80% by weight of at least one perspiration-inhibiting antiperspirant active ingredient in the form of finely divided particles;

(b) from about 0.5% to about 15% by weight of at least one hydrophobized clay;

(c) from about 10% to about 85% by weight of a hydrophobic carrier comprising trans-1-chloro-3,3,3-trifluoropropene (trans-1233zd) and at least one co-carrier wherein: (i) the weight ratio of said trans-1233zd:co-carrier is from about 0.25:1 to about 4:1; (ii) said trans-1233zd and said co-carrier together comprise at least about 40% by weight of said hydrophobic carrier; and (iii) said co-carrier is of a type and present in an amount to provide said formulation with a dry silky feel when sprayed in the form of an aerosol onto the skin of the person; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d).

For the purposes of convenience, antiperspirant formulations according to this paragraph are referred to herein as Formulation 11.

The present invention also provides antiperspirant formulation for delivery in the form of an aerosol to the skin of a person comprising:

(1) non-propellant components comprising:

(a) from about 0.5% to about 80% by weight of at least one perspiration-inhibiting antiperspirant active ingredient in the form of finely divided particles;

(b) from about 0.5% to about 15% by weight of at least one hydrophobized clay;

(c) from about 10% to about 85% by weight of a hydrophobic carrier comprising trans-1-chloro-3,3,3-trifluoropropene (trans-1233zd) and at least one co-carrier comprising one or more of: (i) triglycerides of linear or branched, saturated or unsaturated, C8-30 fatty acids; (ii) esters of at least one C2-C9 monocarboxylic, dicarboxylic or tricarboxylic acid and at least one linear or branched, saturated or unsaturated alcohol having 1 to 30 carbon atoms; and (iii) and C10-C20 iso paraffin, wherein the weight ratio of said trans-1233zd:co-carrier is from about 0.25:1 to about 4:1 and said trans-1233zd and said co-carrier together comprise at least about 40% by weight of said hydrophobic carrier; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d).

For the purposes of convenience, personal care formulations according to this paragraph are referred to herein as Formulation 12.

The present invention also provides an antiperspirant formulation for delivery in the form of an aerosol comprising:

(1) non-propellant components comprising:

(a) from about 1% to about 60% by weight of at least one perspiration-inhibiting antiperspirant active ingredient;

(b) from about 0.5% to about 15% by weight of at least one hydrophobized clay;

(c) from about 10% to about 85% by weight, based on the total weight of the components in the formulation, of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of one or more triglycerides of linear or branched, saturated or unsaturated, C8-30 fatty acids, wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with or separated from said components (a)-(d).

For the purposes of convenience, antiperspirant formulations according to this paragraph are referred to herein as Formulation 13.

The present invention also provides an antiperspirant formulation for delivery in the form of an aerosol comprising:

(1) non-propellant components comprising:

(a) from about 1% to about 60% by weight of at least one perspiration-inhibiting antiperspirant active ingredient;

(b) from about 0.5% to about 15% by weight of at least one hydrophobized clay;

(c) from about 10% to about 85% by weight, based on the total weight of the components in the formulation, of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of one or more triglycerides that are natural oils, wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with or separated from said components (a)-(d).

For the purposes of convenience, antiperspirant formulations according to this paragraph are referred to herein as Formulation 14.

As used herein, “natural oil” refers to oils that are extracted from naturally occurring plant material.

The present invention also provides an antiperspirant formulation for delivery in the form of an aerosol comprising:

(1) non-propellant components comprising:

(a) from about 1% to about 60% by weight of at least one perspiration-inhibiting antiperspirant active ingredient;

(b) from about 0.5% to about 15% by weight of at least one hydrophobized clay;

(c) from about 10% to about 85% by weight, based on the total weight of the components in the formulation, of a hydrophobic carrier comprising at least about 20% by weight of all the carrier components of trans-1-chloro-3,3,3-trifluoropropene (trans-1233zd) and at least about 20% by weight of all the carrier components a first co-carrier selected from the group consisting of consisting of soy bean oil, cottonseed oil, sunflower oil, palm oil, palm kernel oil, linseed oil, almond oil, castor oil, corn oil, olive oil, rapeseed oil, sesame oil, safflower oil, wheat germ oil, peach kernel oil, the liquid components of coconut oil and the combination of two or more of these, wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with or separated from said components (a)-(d).

For the purposes of convenience, antiperspirant formulations according to this paragraph are referred to herein as Formulation 15.

The present invention also provides an antiperspirant formulation for delivery in the form of an aerosol comprising:

(1) non-propellant components comprising:

(a) from about 1% to about 60% by weight of at least one perspiration-inhibiting antiperspirant active ingredient;

(b) from about 0.5% to about 15% by weight of at least one hydrophobized clay;

(c) from about 10% to about 85% by weight, based on the total weight of the components in the formulation, of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of safflower oil, wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with or separated from said components (a)-(d).

For the purposes of convenience, antiperspirant formulations according to this paragraph are referred to herein as Formulation 16.

The present invention also provides an antiperspirant formulation for delivery in the form of an aerosol comprising:

(1) non-propellant components comprising:

(a) from about 1% to about 60% by weight of at least one perspiration-inhibiting antiperspirant active ingredient;

(b) from about 0.5% to about 15% of at least one hydrophobized clay;

(c) from about 10% to about 85% by weight, based on the total weight of the components in the formulation, of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of esters of at least one C2-C7 monocarboxylic, dicarboxylic or tricarboxylic acid and at least one linear or branched, saturated or unsaturated alcohol having 1 to 30 carbon atoms, wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d). For the purposes of convenience, antiperspirant formulations according to this paragraph are referred to herein as Formulation 17.

The present invention also provides an antiperspirant formulation for delivery in the form of an aerosol comprising:

(1) non-propellant components comprising:

(a) from about 1% to about 60% by weight of at least one perspiration-inhibiting antiperspirant active ingredient;

(b) from about 0.5% to about 15% of at least one hydrophobized clay;

(c) from about 10% to about 85% by weight, based on the total weight of the components in the formulation, of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of diesters of at least one C2-C7 dicarboxylic or tricarboxylic acid and at least one linear or branched, saturated or unsaturated alcohol having 4 to 10 carbon atoms wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d). For the purposes of convenience, antiperspirant formulations according to this paragraph are referred to herein as Formulation 18.

The present invention also provides an antiperspirant formulation for delivery in the form of an aerosol comprising:

(1) non-propellant components comprising:

(a) from about 1% to about 60% by weight of at least one perspiration-inhibiting antiperspirant active ingredient;

(b) from about 0.5% to about 15% of at least one hydrophobized clay;

(c) from about 10% to about 85% by weight, based on the total weight of the components in the formulation, of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of diesters of at least one C3-C5 dicarboxylic or tricarboxylic acid and at least one linear or branched, saturated or unsaturated alcohol having 5 to 9 carbon atoms wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d). For the purposes of convenience, antiperspirant formulations according to this paragraph are referred to herein as Formulation 19.

The present invention also provides an antiperspirant formulation for delivery in the form of an aerosol comprising:

(1) non-propellant components comprising:

(a) from about 1% to about 60% by weight of at least one perspiration-inhibiting antiperspirant active ingredient;

(b) from about 0.5% to about 15% of at least one hydrophobized clay;

(c) from about 10% to about 85% by weight, based on the total weight of the components in the formulation, of a hydrophobic carrier comprising at least about 20% by weight of all the carrier components of trans-1-chloro-3,3,3-trifluoropropene (trans-1233zd) and at least about 20% by weight of all the carrier components a first co-carrier consisting of diesters of succinic acid with a linear or branched, saturated or unsaturated alcohol having 7 carbon atoms, wherein the weight ratio of said trans-1233zd:first co-carrier is from about 0.25:1 to about 4:1; and

(d) optionally one or more non-propellant adjuvants, wherein said percentages for components (a)-(d) are based on the total weight of components (a)-(d) in the formulation; and

(2) propellant for imparting motive force to said non-propellant components (a)-(d), wherein said propellant can be interfaced with and/or separated from said components (a)-(d). For the purposes of convenience, antiperspirant formulations according to this paragraph are referred to herein as Formulation 20.

The present invention also provides a drop-in replacement fluid for silicone fluids in personal care compositions, said replacement fluid comprising:

(a) trans-1-chloro-3,3,3-trifluoropropene (trans-1233zd) and

(b) at least one co-carrier wherein: (i) the weight ratio of said trans-1233zd:co-carrier is from about 0.25:1 to about 4:1; and (ii) said co-carrier is of a type and present in an amount to provide said personal care composition with a dry silky feel when sprayed in the form of an aerosol onto the skin of the person and wherein said trans-1233zd and said co-carrier together comprise at least about 90% by weight of said replacement fluid. For the purposes of convenience, antiperspirant formulations according to this paragraph are referred to herein as Replacement Fluid 1.

As the term is used herein, “drop in” replacement fluid means a fluid that can be used in a cosmetic as a complete replacement for all silicone fluids used in an existing formulation. As used herein, complete replacement means that the formulation will be essentially free of silicone fluids and will include the replacement fluid in an amount effective to achieve a cosmetic with substantially the same feel properties as the fluid with the silicone oil.

The present invention also provides a drop-in replacement fluid for silicone fluids in personal care compositions, said replacement fluid comprising:

(a) trans-1-chloro-3,3,3-trifluoropropene (trans-1233zd) and

(b) at least one co-carrier selected from: (i) triglycerides of linear or branched, saturated or unsaturated, C8-30 fatty acids; (ii) esters of at least one C2-C9 monocarboxylic, dicarboxylic or tricarboxylic acid and at least one linear or branched, saturated or unsaturated alcohol having 1 to 30 carbon atoms; and (iii) and C10-C20 iso paraffin and (iv) volatile silicone fluids other than D-4 and D-5, wherein the weight ratio of said trans-1233zd:co-carrier is from about 0.25:1 to about 4:1 and wherein said trans-1233zd and said co-carrier together comprise at least about 90% by weight of said replacement fluid. For the purposes of convenience, antiperspirant formulations according to this paragraph are referred to herein as Replacement Fluid 2.

The present invention also provides a drop-in replacement fluid for silicone fluids in personal care compositions, said replacement fluid comprising:

(a) trans-1-chloro-3,3,3-trifluoropropene (trans-1233zd) and

(b) at least one co-carrier selected from: (i) triglycerides of linear or branched, saturated or unsaturated, C8-30 fatty acids; (ii) esters of at least one C2-C9 monocarboxylic, dicarboxylic or tricarboxylic acid and at least one linear or branched, saturated or unsaturated alcohol having 1 to 30 carbon atoms; and (iii) C10-C20 iso paraffin, wherein the weight ratio of said trans-1233zd:co-carrier is from about 0.25:1 to about 4:1 and wherein said trans-1233zd and said co-carrier together comprise at least about 90% by weight of said replacement fluid. For the purposes of convenience, antiperspirant formulations according to this paragraph are referred to herein as Replacement Fluid 3.

The present invention also provides a drop-in replacement fluid for silicone fluids in personal care compositions, said replacement fluid comprising:

(a) at least about 20% by weight of trans-1-chloro-3,3,3-trifluoropropene (trans-1233zd) and

(b) at least about 20% by weight of at lease one co-carrier a first co-carrier consisting of one or more triglycerides of linear or branched, saturated or unsaturated, C8-C30 fatty acids, wherein the weight ratio of said trans-1233zd:first co-carrier is from about 0.25:1 to about 4:1. For the purposes of convenience, antiperspirant formulations according to this paragraph are referred to herein as Replacement Fluid 4.

The present invention also provides a drop-in replacement fluid for silicone fluids in personal care compositions, said replacement fluid comprising:

(a) at least about 20% by weight of trans-1-chloro-3,3,3-trifluoropropene (trans-1233zd) and

(b) at least about 20% by weight of at least a first co-carrier consisting of one or more triglycerides that are natural oils, wherein the weight ratio of said trans-1233zd:first co-carrier is from about 0.25:1 to about 4:1. For the purposes of convenience, antiperspirant formulations according to this paragraph are referred to herein as Replacement Fluid 5.

The present invention also provides a drop-in replacement fluid for silicone fluids in personal care compositions, said replacement fluid comprising:

(a) at least about 20% by weight of trans-1-chloro-3,3,3-trifluoropropene (trans-1233zd) and

(b) at least about 20% by weight of at least a first co-carrier consisting of soy bean oil, cottonseed oil, sunflower oil, palm oil, palm kernel oil, linseed oil, almond oil, castor oil, corn oil, olive oil, rapeseed oil, sesame oil, safflower oil, wheat germ oil, peach kernel oil, the liquid components of coconut oil and the combination of two or more of these, wherein the weight ratio of said trans-1233zd:first co-carrier is from about 0.25:1 to about 4:1. For the purposes of convenience, antiperspirant formulations according to this paragraph are referred to herein as Replacement Fluid 6.

The present invention also provides a drop-in replacement fluid for silicone fluids in personal care compositions, said replacement fluid comprising:

(a) at least about 20% by weight of trans-1-chloro-3,3,3-trifluoropropene (trans-1233zd) and

(b) at least about 20% by weight of at least a first co-carrier consisting of safflower oil, wherein the weight ratio of said trans-1233zd:first co-carrier is from about 0.25:1 to about 4:1. For the purposes of convenience, antiperspirant formulations according to this paragraph are referred to herein as Replacement Fluid 7.

The present invention also provides a drop-in replacement fluid for silicone fluids in personal care compositions, said replacement fluid comprising:

(a) at least about 20% by weight of trans-1-chloro-3,3,3-trifluoropropene (trans-1233zd) and

(b) at least about 20% by weight of at least a first co-carrier consisting of esters of at least one C2-C7 monocarboxylic, dicarboxylic or tricarboxylic acid and at least one linear or branched, saturated or unsaturated alcohol having 1 to 30 carbon atoms, wherein the weight ratio of said trans-1233zd:first co-carrier is from about 0.25:1 to about 4:1. For the purposes of convenience, antiperspirant formulations according to this paragraph are referred to herein as Replacement Fluid 8.

The present invention also provides a drop-in replacement fluid for silicone fluids in personal care compositions, said replacement fluid comprising:

(a) at least about 20% by weight of trans-1-chloro-3,3,3-trifluoropropene (trans-1233zd) and

(b) at least about 20% by weight of at least a first co-carrier consisting of diesters of at least one C2-C7 dicarboxylic or tricarboxylic acid and at least one linear or branched, saturated or unsaturated alcohol having 4 to 10 carbon atoms, wherein the weight ratio of said trans-1233zd:first co-carrier is from about 0.25:1 to about 4:1. For the purposes of convenience, antiperspirant formulations according to this paragraph are referred to herein as Replacement Fluid 9.

The present invention also provides a drop-in replacement fluid for silicone fluids in personal care compositions, said replacement fluid comprising:

(a) at least about 20% by weight of trans-1-chloro-3,3,3-trifluoropropene (trans-1233zd) and

(b) at least about 20% by weight of at least a first co-carrier consisting of diesters of at least one C3-C5 dicarboxylic or tricarboxylic acid and at least one linear or branched, saturated or unsaturated alcohol having 5 to 9 carbon atoms, wherein the weight ratio of said trans-1233zd:first co-carrier is from about 0.25:1 to about 4:1. For the purposes of convenience, antiperspirant formulations according to this paragraph are referred to herein as Replacement Fluid 10.

The present invention also provides a drop-in replacement fluid for silicone fluids in antiperspirant formulations, including the use of each of Replacement Fluids 1-10 as a drop-in replacement for D-4 or D-5 in antiperspirant formulations.

The present invention also provides methods for applying a personal care formulation in the form of an aerosol to a person comprising:

(A) providing in a container a personal care composition of the present invention, including each of Formulations 1-10; and

(B) applying at least said non-propellant components of said personal care composition to said person.

The present invention also provides methods for applying an antiperspirant formulation in the form of an aerosol to a person comprising:

(A) providing in a container a personal care composition of the present invention, including each of Formulations 11-20; and

(B) applying at least said non-propellant components of said personal care composition to said person.

The present invention thus also provides a method for delivering a personal care formulation to the skin of a human comprising:

(a) providing a container for containing an personal care formulation of the present invention, including each of Formulations 1-10;

(b) and controllably discharging in the form of an aerosol at least a portion of said formulation in said container for the production of a spray of containing at least a portion of said personal care formulation.

The present invention thus also provides a method for delivering an antiperspirant to the skin of a human comprising:

(a) providing a container for containing an antiperspirant formulation of the present invention, including each of Formulations 11-20; and

(b) controllably discharging in the form of an aerosol at least a portion of said formulation in said container for the production of a spray of containing at least a portion of said antiperspirant formulation.

The present invention thus also provides a device for delivering a personal care formulation to the skin of a human comprising:

(a) a container for containing an personal care formulation of the present invention, including each of Formulations 1-10;

(b) a valve for controllably discharging in the form of an aerosol at least a portion of said formulation in said container for the production of a spray of containing at least a portion of said personal care formulation. For the purposes of convenience, devices according to this paragraph are referred to herein as Device 1.

The present invention thus also provides a device for delivering an antiperspirant to the skin of a human comprising:

(a) a container for containing an antiperspirant formulation of the present invention, including each of Formulations 11-20; and

(b) a valve for controllably discharging in the form of an aerosol at least a portion of said formulation in said container for the production of a spray of containing at least a portion of said antiperspirant formulation. For the purposes of convenience, devices according to this paragraph are referred to herein as Device 2. An example of a device useful in accordance with the present invention is disclose in U.S. Pat. No. 5,082,652, which is incorporated herein by reference.

### EXAMPLES

### Comparative Example 1

A formulation representative of a commercial antiperspirant product as described in Table C1 below is obtained and tested:

TABLE C1

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3 | D-5 | Hydrophobic | 4.8 | 30 |
| | | carrier | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

The formulation has spray characteristics representative of commercially available antiperspirant aerosol products based on D-5 and has an acceptable dry, silky feel on the skin representative of commercially available antiperspirant aerosol products based on D-5.

### Comparative Example 2

A formulation the same as shown in Table C1 was prepared except trans-1233zd is used in place of D-5 on an equal weight basis as indicated in Table C2 below:

TABLE C2

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3 | trans-1233zd | Hydrophobic | 4.8 | 30 |
| | | carrier | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

The formulation was prepared by first adding the indicated amounts of components 1, 2 and 4 into a beaker and then mixing those components until uniform using a rotor stator mixer. The mixture builds viscosity as mixing continued. Then component 5 was added as mixing was continued and viscosity slowly continues to increase as mixing continues for an additional five (5) minutes. The mixture of components 1, 2, 4 and 5 from the beaker was then added into a sealable container with trans-1233zd that had been chilled to a temperature below 19° C. The vessel was then sealed and mixing continued until the contents were uniform, and then the mixture is allowed to equilibrate for about 30-60 minutes. The liquid concentrate mixture thus produced is added into a sealable aluminum spray can, the can is sealed and then the propellant (component 6) is charged into the can.

The formulation is tested by spraying onto the skin of a user. While the formulation has generally acceptable spray characteristics similar to representative commercially available antiperspirant aerosol products based on D-5 as described in Comparative Example C1, it has a feel that is much dryer on the skin than commercially available D-5 antiperspirant aerosol products based on the results from Example C1.

### Example 1

A formulation the same as shown in Table C1 was prepared except a 25:75 weight ratio mixture of trans-1233zd and safflower oil is used in place of D-5 on an equal weight basis as indicated in Table 1 below:

TABLE 1

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3A | trans-1233zd | Hydrophobic | 1.2 | 7.5 |
| | | carrier | | |
| 3B | Safflower oil | Hydrophobic | 3.6 | 22.5 |
| | | carrier | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

The formulation was prepared by first adding the indicated amounts of components 1, 2, 3B and 4 into a beaker and then mixing those components until uniform using a rotor stator mixer. The mixture built viscosity as mixing continued. Then component 5 was added as mixing was continued and viscosity slowly continued to increase as mixing continued for an additional five (5) minutes. The mixture of components 1, 2, 3B, 4 and 5 from the beaker was then added into a sealable container with component 3A (trans-1233zd) that had been chilled to a temperature below 19° C. The vessel was then sealed and mixing continued until the contents were uniform, and then the mixture is allowed to equilibrate for about 30-60 minutes. The liquid concentrate mixture thus produced is added into a sealable aluminum spray can, the can is sealed and then the propellant (component 6) is charged into the can.

The formulation is tested by spraying onto the skin of a user. The formulation has acceptable spray characteristics similar to representative commercially available antiperspirant aerosol products based on D-5 as described in Comparative Example C1 and it has a feel on the skin that is as good as or better than the commercially available D-5 antiperspirant aerosol products based on comparison to the results from Example C1.

### Example 2

A formulation the same as shown in Table C1 was prepared except a 50:50 weight ratio mixture of trans-1233zd and safflower oil is used in place of D-5 on an equal weight basis as indicated in Table 2 below:

TABLE 2

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3A | trans-1233zd | Hydrophobic | 2.4 | 15 |
| | | carrier | | |
| 3B | Safflower oil | Hydrophobic | 2.4 | 15 |
| | | carrier | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

The formulation was prepared by first adding the indicated amounts of components 1, 2, 3B and 4 into a beaker and then mixing those components until uniform using a rotor stator mixer. The mixture built viscosity as mixing continued. Then component 5 was added as mixing was continued and viscosity slowly continued to increase as mixing continued for an additional five (5) minutes. The mixture of components 1, 2, 3B, 4 and 5 from the beaker was then added into a sealable container with component 3A (trans-1233zd) that had been chilled to a temperature below 19° C. The vessel was then sealed and mixing continued until the contents were uniform, and then the mixture is allowed to equilibrate for about 30-60 minutes. The liquid concentrate mixture thus produced is added into a sealable aluminum spray can, the can is sealed and then the propellant (component 6) is charged into the can.

The formulation is tested by spraying onto the skin of a user. The formulation has acceptable spray characteristics similar to representative commercially available antiperspirant aerosol products based on D-5 as described in Comparative Example C1 and it has a feel on the skin that is as good as or better than the commercially available D-5 antiperspirant aerosol products based on comparison to the results from Example C1.

### Example 3

A formulation the same as shown in Table C1 was prepared except a 75:25 weight ratio mixture of trans-1233zd and safflower oil is used in place of D-5 on an equal weight basis as indicated in Table 3 below:

TABLE 3

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3A | trans-1233zd | Hydrophobic | 3.6 | 22.5 |
| | | carrier | | |
| 3B | Safflower oil | Hydrophobic | 1.2 | 7.5 |
| | | carrier | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

The formulation was prepared by first adding the indicated amounts of components 1, 2, 3B and 4 into a beaker and then mixing those components until uniform using a rotor stator mixer. The mixture built viscosity as mixing continued. Then component 5 was added as mixing was continued and viscosity slowly continued to increase as mixing continued for an additional five (5) minutes. The mixture of components 1, 2, 3B, 4 and 5 from the beaker was then added into a sealable container with component 3A (trans-1233zd) that had been chilled to a temperature below 19° C. The vessel was then sealed and mixing continued until the contents were uniform, and then the mixture is allowed to equilibrate for about 30-60 minutes. The liquid concentrate mixture thus produced is added into a sealable aluminum spray can, the can is sealed and then the propellant (component 6) is charged into the can.

The formulation is tested by spraying onto the skin of a user. The formulation has acceptable spray characteristics similar to representative commercially available antiperspirant aerosol products based on D-5 as described in Comparative Example C1 and it has a feel on the skin that is as good as or better than the commercially available D-5 antiperspirant aerosol products based on comparison to the results from Example C1.

### Comparative Example 3

A formulation the same as shown in Table C1 was prepared except safflower oil is used in place of D-5 on an equal weight basis as indicated in Table C3 below:

TABLE C3

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3 | Safflower oil | Hydrophobic | 4.8 | |
| | | carrier | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

The formulation was prepared by first adding the indicated amounts of components 1, 2, 3 and 4 into a beaker and then mixing those components until uniform using a rotor stator mixer. The mixture built viscosity as mixing continued. Then component 5 was added as mixing was continued and viscosity slowly continued to increase as mixing continued for an additional five (5) minutes. The mixture of components 1, 2, 3, 4 and 5 from the beaker was then added into a sealable container. The vessel was then sealed and mixing continued until the contents were uniform, and then the mixture is allowed to equilibrate for about 30-60 minutes. The liquid concentrate mixture thus produced is added into a sealable aluminum spray can, the can is sealed and then the propellant (component 6) is charged into the can.

The formulation is tested by spraying onto the skin of a user. The formulation has spray characteristics substantially inferior to the representative commercially available antiperspirant aerosol products based on D-5 as described in Comparative Example C1, that is, the spray was found to be wet and to have a high level of liquidity.

### Comparative Example 4—D-5 Replaced by Diheptylsuccinate Lex-Feel N-20

A formulation the same as shown in Table C1 was prepared except diheptyl succinate based carrier sold under the trade designation Lex-Feel N-20 is used in place of D-5 on an equal weight basis as indicated in Table C4 below:

TABLE C4

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3 | Lex-feel N-20 | Hydrophobic | 4.8 | 30 |
| | (diheptyl | carrier | | |
| | succinate with | | | |
| | capryloyl | | | |
| | glycerin/ | | | |
| | sebacic acid | | | |
| | copolymer) | | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

The formulation was prepared by first adding the indicated amounts of components 1, 2, 3 and 4 into a beaker and then mixing those components until uniform using a rotor stator mixer. The mixture built viscosity as mixing continued. Then component 5 was added as mixing was continued and viscosity slowly continued to increase as mixing continued for an additional five (5) minutes. The mixture of components 1, 2, 3, 4 and 5 from the beaker was then added into a sealable container. The vessel was then sealed and mixing continued until the contents were uniform, and then the mixture is allowed to equilibrate for about 30-60 minutes. The liquid concentrate mixture thus produced is added into a sealable aluminum spray can, the can is sealed and then the propellant (component 6) is charged into the can.

The formulation is tested by spraying onto the skin of a user. The formulation has spray characteristics substantially inferior to the representative commercially available antiperspirant aerosol products based on D-5 as described in Comparative Example C1, that is, the spray was found to have some clumping of the powder. In addition, the feel of the formulation was substantially inferior to the representative commercially available antiperspirant aerosol products based on D-5 as described in Comparative Example C1, that is, the formulation had a felt wet and was found to have an inferior high level of liquidity.

### Example 4—D-5 Replaced by 50:50 Trans1233zd and Lex-Feel N-20

A formulation the same as shown in Table C1 was prepared except a 50:50 weight ratio mixture of trans-1233zd and Lex-feel N-20 is used in place of D-5 on an equal weight basis as indicated in Table 4 below:

TABLE 4

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3A | trans-1233zd | Hydrophobic | 2.4 | 15 |
| | | carrier | | |
| 3B | Lex-feel N-20 | Hydrophobic | 2.4 | 15 |
| | (diheptyl | carrier | | |
| | succinate with | | | |
| | capryloyl | | | |
| | glycerin/ | | | |
| | sebacic acid | | | |
| | copolymer) | | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

The formulation was prepared by first adding the indicated amounts of components 1, 2, 3B and 4 into a beaker and then mixing those components until uniform using a rotor stator mixer. The mixture built viscosity as mixing continued. Then component 5 was added as mixing was continued and viscosity slowly continued to increase as mixing continued for an additional five (5) minutes. The mixture of components 1, 2, 3B, 4 and 5 from the beaker was then added into a sealable container with component 3A (trans-1233zd) that had been chilled to a temperature below 19° C. The vessel was then sealed and mixing continued until the contents were uniform, and then the mixture is allowed to equilibrate for about 30-60 minutes. The liquid concentrate mixture thus produced is added into a sealable aluminum spray can, the can is sealed and then the propellant (component 6) is charged into the can.

The formulation is tested by spraying onto the skin of a user. The formulation has acceptable spray characteristics similar to representative commercially available antiperspirant aerosol products based on D-5 as described in Comparative Example C1 and it has a feel on the skin that is as good as or better than the commercially available D-5 antiperspirant aerosol products based on comparison to the results from Example C1.

### Comparative Example 5—D-5 Replaced by Diheptylsuccinate in Lex-Feel N-7

A formulation the same as shown in Table C1 was prepared except diheptyl succinate based carrier sold under the trade designation Lex-Feel N-7 (with a lower viscosity than N-20) is used in place of D-5 on an equal weight basis as indicated in Table C5 below:

TABLE C5

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3 | Lex-feel N-7 | Hydrophobic | 4.8 | 30 |
| | (diheptyl | carrier | | |
| | succinate with | | | |
| | capryloyl | | | |
| | glycerin/ | | | |
| | sebacic acid | | | |
| | copolymer) | | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

The formulation was prepared by first adding the indicated amounts of components 1, 2, 3 and 4 into a beaker and then mixing those components until uniform using a rotor stator mixer. The mixture built viscosity as mixing continued. Then component 5 was added as mixing was continued and viscosity slowly continued to increase as mixing continued for an additional five (5) minutes. The mixture of components 1, 2, 3, 4 and 5 from the beaker was then added into a sealable container. The vessel was then sealed and mixing continued until the contents were uniform, and then the mixture is allowed to equilibrate for about 30-60 minutes. The liquid concentrate mixture thus produced is added into a sealable aluminum spray can, the can is sealed and then the propellant (component 6) is charged into the can.

The formulation is tested by spraying onto the skin of a user. The formulation has spray characteristics similar to the representative commercially available antiperspirant aerosol products based on D-5 as described in Comparative Example C1. However, in terms of feel on the skin the formulation was substantially inferior to the representative commercially available antiperspirant aerosol products based on D-5 as described in Comparative Example C1, that is, the formulation felt very oily.

### Example 5—D-5 Replaced by 50:50 Trans1233zd and Lex-Feel N-7

A formulation the same as shown in Table C1 was prepared except a 50:50 weight ratio mixture of trans-1233zd and Lex-feel N-7 is used in place of D-5 on an equal weight basis as indicated in Table 5 below:

TABLE 5

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3A | trans-1233zd | Hydrophobic | 2.4 | 15 |
| | | carrier | | |
| 3B | Lex-feel N-7 | Hydrophobic | 2.4 | 15 |
| | (diheptyl | carrier | | |
| | succinate with | | | |
| | capryloyl | | | |
| | glycerin/ | | | |
| | sebacic acid | | | |
| | copolymer) | | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

The formulation was prepared by first adding the indicated amounts of components 1, 2, 3B and 4 into a beaker and then mixing those components until uniform using a rotor stator mixer. The mixture built viscosity as mixing continued. Then component 5 was added as mixing was continued and viscosity slowly continued to increase as mixing continued for an additional five (5) minutes. The mixture of components 1, 2, 3B, 4 and 5 from the beaker was then added into a sealable container with component 3A (trans-1233zd) that had been chilled to a temperature below 19° C. The vessel was then sealed and mixing continued until the contents were uniform, and then the mixture is allowed to equilibrate for about 30-60 minutes. The liquid concentrate mixture thus produced is added into a sealable aluminum spray can, the can is sealed and then the propellant (component 6) is charged into the can.

The formulation is tested by spraying onto the skin of a user. The formulation has acceptable spray characteristics similar to representative commercially available antiperspirant aerosol products based on D-5 as described in Comparative Example C1 and it has a feel on the skin that is as good as or better than the commercially available D-5 antiperspirant aerosol products based on comparison to the results from Example C1.

### Example 5

A formulation the same as shown in Table C1 was prepared except a 75:25 weight ratio mixture of trans-1233zd and safflower oil is used in place of D-5 on an equal weight basis as indicated in Table 3 below:

TABLE 3

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3A | trans-1233zd | Hydrophobic | 3.6 | 22.5 |
| | | carrier | | |
| 3B | Safflower oil | Hydrophobic | 1.2 | 7.5 |
| | | carrier | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

The formulation was prepared by first adding the indicated amounts of components 1, 2, 3B and 4 into a beaker and then mixing those components until uniform using a rotor stator mixer. The mixture built viscosity as mixing continued. Then component 5 was added as mixing was continued and viscosity slowly continued to increase as mixing continued for an additional five (5) minutes. The mixture of components 1, 2, 3B, 4 and 5 from the beaker was then added into a sealable container with component 3A (trans-1233zd) that had been chilled to a temperature below 19° C. The vessel was then sealed and mixing continued until the contents were uniform, and then the mixture is allowed to equilibrate for about 30-60 minutes. The liquid concentrate mixture thus produced is added into a sealable aluminum spray can, the can is sealed and then the propellant (component 6) is charged into the can.

The formulation is tested by spraying onto the skin of a user. The formulation has acceptable spray characteristics similar to representative commercially available antiperspirant aerosol products based on D-5 as described in Comparative Example C1 and it has a feel on the skin that is as good as or better than the commercially available D-5 antiperspirant aerosol products based on comparison to the results from Example C1.

### Example 6

A formulation the same as shown in Table C1 is prepared except a 25:75 weight ratio mixture of trans-1233zd and each of the components identified in Table 6B was is used in place of D-5 on an equal weight basis as indicated in Table 6A below:

TABLE 6A

| | | | Wt % | Wt % |
|---|---|---|---|---|
| Com- | | | Based of | of non-pro- |
| ponent | Com- | | All Com- | pellant com- |
| No. | ponent | Function | ponents | ponents |
| 1 | Bentone Gel | | 1.3 | 8.13 |
| | GTCCV | | | |
| | consisting of: | | | |
| | stearalkonium | hydrophobized | | |
| | hectorite | clay | | |
| | propylene | activator for | | |
| | carbonate | hydrophobized | | |
| | | clay | | |
| | caprylic/capric | hydrophobic | | |
| | triglyceride | carrier | | |
| 2 | C12-C15 alkyl | Hydrophobic | 2.9 | 18.13 |
| | benzoate | carrier | | |
| 3A | trans-1233zd | Hydrophobic | 1.2 | 7.5 |
| | | carrier | | |
| 3B | Seperately | Hydrophobic | 3.6 | 22.5 |
| | each of | carrier | | |
| | components | | | |
| | 1-22 as per | | | |
| | Table 6B | | | |
| | below | | | |
| 4 | Fragrance | | 1.0 | 6.25 |
| 5 | Aluminum | Antiperspirant | 6 | 37.5 |
| | chlorohydrate | active | | |
| 6 | A-46 | Propellant | 84 | |
| Total | | | 100 | 100 |

TABLE 6B

| Compo- | |
|---|---|
| nent No. | Component Description |
| 1 | a triglyceride of linear or branched, saturated or unsaturated, |
| | C₈₋₃₀ fatty acids |
| 2A | an ester of at least one C₂-C₉ monocarboxylic acid and a |
| | linear saturated alcohol having 1 to 30 carbon atoms |
| 2B | an ester of at least one C₂-C₉ monocarboxylic acid and a |
| | linear unsaturated alcohol having 1 to 30 carbon atoms |
| 2C | an ester of at least one C₂-C₉ monocarboxylic acid and a |
| | branched, saturated alcohol having 1 to 30 carbon atoms |
| 2D | an ester of at least one C₂-C₉ monocarboxylic acid and |
| | branched, unsaturated alcohol having 1 to 30 carbon atoms |
| 3A | an ester of at least one C₂-C₉ dicarboxylic acid and a linear |
| | saturated alcohol having 1 to 30 carbon atoms |
| 3B | an ester of at least one C₂-C₉ dicarboxylic acid and a linear |
| | unsaturated alcohol having 1 to 30 carbon atoms |
| 3C | an ester of at least one C₂-C₉ dicarboxylic acid and a |
| | branched, saturated alcohol having 1 to 30 carbon atoms |
| 3D | an ester of at least one C₂-C₉ dicarboxylic acid and |
| | branched, unsaturated alcohol having 1 to 30 carbon atoms |
| 4A | an ester of at least one C₂-C₉ tricarboxylic acid and a linear |
| | saturated alcohol having 1 to 30 carbon atoms |
| 4B | an ester of at least one C₂-C₉ tricarboxylic acid and a linear |
| | unsaturated alcohol having 1 to 30 carbon atoms |
| 5C | an ester of at least one C₂-C₉ tricarboxylic acid and a |
| | branched, saturated alcohol having 1 to 30 carbon atoms |
| 5D | an ester of at least one C₂-C₉ tricarboxylic acid and |
| | branched, unsaturated alcohol having 1 to 30 carbon atoms |
| 6A | C₁₀ iso paraffin |
| 6B | C₁₁ iso paraffin |
| 6C | C₁₂ iso paraffin |
| 6D | C₁₃ iso paraffin |
| 6E | C₁₄ iso paraffin |
| 6F | C₁₅ iso paraffin |
| 6G | C₁₆ iso paraffin |
| 6H | C₁₇ iso paraffin |
| 6I | C₁₈ iso paraffin |
| 6J | C₁₉ iso paraffin |
| 6K | C₂₀ iso paraffin |
| 7 | volatile silicone fluid other than D-4 and D-5 |
| 8 | Triglyceride - natural oil |
| 9 | soy bean oil |
| 10 | cottonseed oil |
| 11 | sunflower oil |
| 12 | palm oil |
| 13 | palm kernel oil |
| 14 | linseed oil |
| 15 | almond oil |
| 16 | castor oil |
| 17 | corn oil |
| 18 | rapeseed oil |
| 19 | sesame oil |
| 20 | wheat germ oil |
| 21 | peach kernel oil |
| 22 | liquid components of coconut oil |

The formulation is prepared by first adding the indicated amounts of components 1, 2, 3B and 4 into a beaker and then mixing those components until uniform using a rotor stator mixer. The mixture built viscosity as mixing continued. Then component 5 is added as mixing is continued and viscosity slowly continued to increase as mixing continued for an additional five (5) minutes. The mixture of components 1, 2, 3B, 4 and 5 from the beaker is then added into a sealable container with component 3A (trans-1233zd) that had been chilled to a temperature below 19° C. The vessel is then sealed and mixing continued until the contents are uniform, and then the mixture is allowed to equilibrate for about 30-60 minutes. The liquid concentrate mixture thus produced is added into a sealable aluminum spray can, the can is sealed and then the propellant (component 6) is charged into the can.

The formulation is tested by spraying onto the skin of a user. The formulation has acceptable spray characteristics similar to representative commercially available antiperspirant aerosol products based on D-5 as described in Comparative Example C1 and it has a feel on the skin that is as good as or better than the commercially available D-5 antiperspirant aerosol products based on comparison to the results from Example C1.

What is claimed is:

1. A personal care formulation for delivery to the skin of a person in the form of an aerosol comprising:
(1) non-propellant components comprising:
(a) from about 0.5% to about 80% by weight of at least one active ingredient in the form of finely divided particles;
(b) from about 0.5% to about 15% by weight of at least one hydrophobized clay;
(c) from about 10% to about 85% by weight of a hydrophobic carrier comprising trans-1-chloro-3,3,3-trifluoropropene (trans-1233zd) and at least one co-carrier wherein: (i) the weight ratio of said trans-1233zd:co-carrier is from about 0.25:1 to about 4:1; (ii) said trans-1233zd and said co-carrier together comprise at least about 40% by weight of said hydrophobic carrier; and
(d) optionally one or more non-propellant adjuvants, wherein said percentages for components (a)-(d) are based on the total weight of components (a)-(d) in the formulation; and
(2) propellant for imparting motive force to said non-propellant components (a)-(d), wherein said propellant can be interfaced with and/or separated from said components (a)-(d).

2. The formulation of claim 1 wherein said at least one co-carrier comprises one or more of: (i) triglycerides of linear or branched, saturated or unsaturated, C8-30 fatty acids; (ii) esters of at least one C₂-C₉ monocarboxylic, dicarboxylic or tricarboxylic acid and at least one linear or branched, saturated or unsaturated alcohol having 1 to 30 carbon atoms; (iii) C₁₀-C₂₀ iso paraffin; (iv) volatile silicone fluids other than D-4 and D-5, and combinations of two or more of these.

3. The formulation of claim 1 wherein said at least one active ingredient from about 1% to about 60% by weight of the formulation.

4. The formulation of claim 1 wherein said co-carrier is of a type and present in an amount to provide said formulation with a dry silky feel when sprayed in the form of an aerosol onto the skin of the person.

5. The formulation of claim 1 wherein said at least one co-carrier comprises at least about 20% by weight of all the carrier components and consists essentially of one or more triglycerides of linear or branched, saturated or unsaturated, C₈₋₃₀ fatty acids and combinations of two or more of these.

6. The formulation of claim 5 wherein said hydrophobic carrier comprises at least about 20% by weight of all the carrier components of trans-1-chloro-3,3,3-trifluoropropene (trans-1233zd).

7. The formulation of claim 1 wherein said at least one co-carrier consists essentially of one or more triglycerides that are natural oils.

8. The formulation of claim 1 wherein said at least one co-carrier is selected from the group consisting of soy bean oil, cottonseed oil, sunflower oil, palm oil, palm kernel oil, linseed oil, almond oil, castor oil, corn oil, olive oil, rapeseed oil, sesame oil, safflower oil, wheat germ oil, peach kernel oil, the liquid components of coconut oil and the combination of two or more of these.

8. The formulation of claim 1 wherein said at least one co-carrier comprises safflower oil.

9. The formulation of claim 1 wherein said at least one co-carrier comprises safflower oil present in an amount of at least about 20% by weight based on all the carrier components.

10. The formulation of claim 1 in the form of an antiperspirant formulation for delivery to the skin of a person in the form of an aerosol and wherein said active ingredient comprises perspiration-inhibiting antiperspirant active ingredient.

11. A drop-in replacement fluid for silicone fluids in personal care compositions, said replacement fluid comprising:
(a) trans-1-chloro-3,3,3-trifluoropropene (trans-1233zd) and
(b) at least one co-carrier wherein: (i) the weight ratio of said trans-1233zd:co-carrier is from about 0.25:1 to about 4:1; and (ii) said trans-1233zd and said co-carrier together comprise at least about 90% by weight of said replacement fluid.

12. The drop-in replacement fluid of claim 11 wherein said co-carrier is of a type and present in an amount to provide said personal care composition with a dry silky feel when sprayed in the form of an aerosol onto the skin of the person.

13. The drop-in replacement fluid of claim 11 wherein said at least one co-carrier comprises one or more of: (i) triglycerides of linear or branched, saturated or unsaturated, C₈₋₃₀ fatty acids; (ii) esters of at least one C₂-C₉ monocarboxylic, dicarboxylic or tricarboxylic acid and at least one linear or branched, saturated or unsaturated alcohol having 1 to 30 carbon atoms; (iii) C₁₀-C₂₀ iso paraffin; (iv) volatile silicone fluids other than D-4 and D-5, and combinations of two or more of these.

14. The drop-in replacement fluid of claim 11 wherein said co-carrier is of a type and present in an amount to provide said formulation with a dry silky feel when sprayed in the form of an aerosol onto the skin of the person.

15. The drop-in replacement fluid of claim 11 wherein said at least one co-carrier: (i) comprises at least about 20% by weight of all the carrier components; and (2) consists essentially of one or more triglycerides of linear or branched, saturated or unsaturated, C₈₋₃₀ fatty acids and combinations of two or more of these.

16. The drop-in replacement fluid of claim 15 wherein said replacement fluid comprises at least about 20% by weight of trans-1-chloro-3,3,3-trifluoropropene (trans-1233zd).

17. The drop-in replacement fluid of claim 11 wherein said at least one co-carrier consists essentially of one or more triglycerides that are natural oils.

18. The drop-in replacement fluid of claim 11 wherein said at least one co-carrier is selected from the group consisting of soy bean oil, cottonseed oil, sunflower oil, palm oil, palm kernel oil, linseed oil, almond oil, castor oil, corn oil, olive oil, rapeseed oil, sesame oil, safflower oil, wheat germ oil, peach kernel oil, the liquid components of coconut oil and the combination of two or more of these.

19. The drop-in replacement fluid of claim 11 wherein said at least one co-carrier comprises safflower oil.

20. The drop-in replacement fluid of claim 11 wherein said at least one co-carrier is present in an amount of at least about 20% by weight based on all the carrier components.
